(19) United States
(12) Patent Application Publication
(10) Pub. No.: US 2023/0059691 A1
(43) Pub. Date: Feb. 23, 2023

(54) Silent Speech and Silent Listening System

(71) Applicant: Philip R. Kennedy, Duluth, GA (US)

(72) Inventor: Philip R. Kennedy, Duluth, GA (US)

(21) Appl. No.: 17/795,502

(22) PCT Filed: Feb. 10, 2021
(86) PCT No.: PCT/US2021/017388

§ 371 Date: Jul. 26, 2022

Related U.S. Application Data
(60) Provisional application No. 62/975,014, filed on Feb. 11, 2020.

(51) Int. Cl. G10L 13/027; G06F 3/01; A61B 5/00

(52) U.S. Cl. CPC G10L 13/027; G06F 3/015; A61B 5/6868

(57) ABSTRACT

A silent communication system (100) for communication between a first person (22) having a speech motor cortex (12) and a second person (24) employs a speech motor cortex neural sensing device (120) and senses speech neural impulses generated by the first person (22) who is generating motor neural potentials corresponding to speech. A wireless transmission device (122) is disposed on the first person (22) and communicates with the speech motor cortex neural sensing device (120). The wireless transmission device (122) generates a radio frequency signal corresponding to the speech neural impulses. A speech generating device (130) is disposed on he second person (24) and is responsive to the radio frequency signal. The speech generating device (130) generates a reconstruction of the speech of the first person (22) that is audibly perceptible by the second person (24).

### CROSS-REFERENCE TO RELATED APPLICATION(S)

This application claims the benefit of U.S. Provisional Patent Application Ser. No. 62/975,014, filed Feb. 11, 2020, the entirety of which is hereby incorporated herein by reference.

### BACKGROUND OF THE INVENTION

### 1. Field of the Invention

The present invention relates to communication systems and, more specifically, to a system that allows a first person to generate speech silently and that allows a second person to perceive the speech in real time.

### 2. Description of the Related Art

Clandestine tactics often require silent communication. For example, special forces operators often need to communicate silently during covert missions. Typically, they use hand signals to communicate. However, the number of hand signals that they use typically covey limited amounts of information. Also, hand signals are hard to perceive at night.

Therefore, there is a need for a system that allows individuals to communicate silently by generating speech motor potentials.

### SUMMARY OF THE INVENTION

The disadvantages of the prior art are overcome by the present invention which, in one aspect, is a silent communication system for communication between a first person having a speech motor cortex and a second person. A speech motor cortex neural sensing device is configured to sense speech neural impulses generated by the first person when the first person is generating motor neural potentials corresponding to speech. A wireless transmission device is configured to be disposed on the first person and is in communication with the speech motor cortex neural sensing device. The wireless transmission device generates a radio frequency signal corresponding to the speech neural impulses. A speech generating device is disposed on the second person and is responsive to the radio frequency signal. The speech generating device is configured to generate a reconstruction of the speech of the first person that is audibly perceptible by the second person.

In another aspect, the invention is a method of communicating silently between a first person having a speech motor cortex and a second person, in which speech neural impulses are sensed from the speech motor cortex of the first person when the person generates motor neural potentials corresponding to speech. An electrical signal corresponding to synthesized speech is generated using the speech neural impulses as input. The electrical signal is modulated onto a radio frequency signal. The radio frequency signal is transmitted. The radio frequency signal is received. Reconstructed speech that is audibly perceptible by the second person is generated from the radio frequency signal.

In yet another aspect, the invention is a method of communicating silently between a first person having a speech motor cortex and a second person, in which speech neural impulses are sensed from the speech motor cortex of the first person when the person generates motor neural potentials corresponding to speech. An electrical signal corresponding to the speech neural impulses is generated. The electrical signal is modulated onto a radio frequency signal. The radio frequency signal is transmitted and received. The radio frequency signal is demodulated. The speech neural impulses are decoded into phones, phonemes, words or phrases. Reconstructed speech that is audibly perceptible by the second person is generated from the phones, phonemes, words or phrases from the radio frequency signal.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram showing one representative embodiment of a silent speech generating system.

FIG. 2A is a schematic diagram showing an embodiment of a silent speech generating system and earpiece-type receiving system.

FIG. 2B is a schematic diagram showing an embodiment of a silent speech generating system and implant-type receiving system.

FIG. 2C is a schematic diagram showing an embodiment of a silent speech generating system and cellular telephone-type receiving system.

FIG. 3 is a schematic diagram showing electronic components employed in a silent speech generating system.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

A “phone” is any distinct speech sound or gesture, regardless of whether the exact sound is critical to the meanings of words. A “phoneme” is a speech sound in a given language that, if swapped with another phoneme, could change one word to another. Phones are absolute and are not specific to any language, but phonemes are usually discussed in reference to specific languages.

One method of detecting speech from brain activity is disclosed in U.S. Pat. No. 7,275,035 and is incorporated herein by reference. One embodiment of a neural electrode array is disclosed in U.S. Publication No. US-2006-0224212-A1 and is incorporated herein by reference. One example of an implantable neural electrode is disclosed in U.S. Pat. No. 10,5757,50 and is incorporated herein by reference.

One embodiment includes a communication system whose electronics are completely implanted under the scalp, with recording electrodes just millimeters under the surface of the speech cortex, wirelessly powered and transmitted using FM transmission. Communication from a colleague can be received using a hearing aid embedded in the mastoid bone. Groups of individuals (such as members of a military patrol or a special warfare team) can be implanted so that communications are completely imperceptible to outside observers.

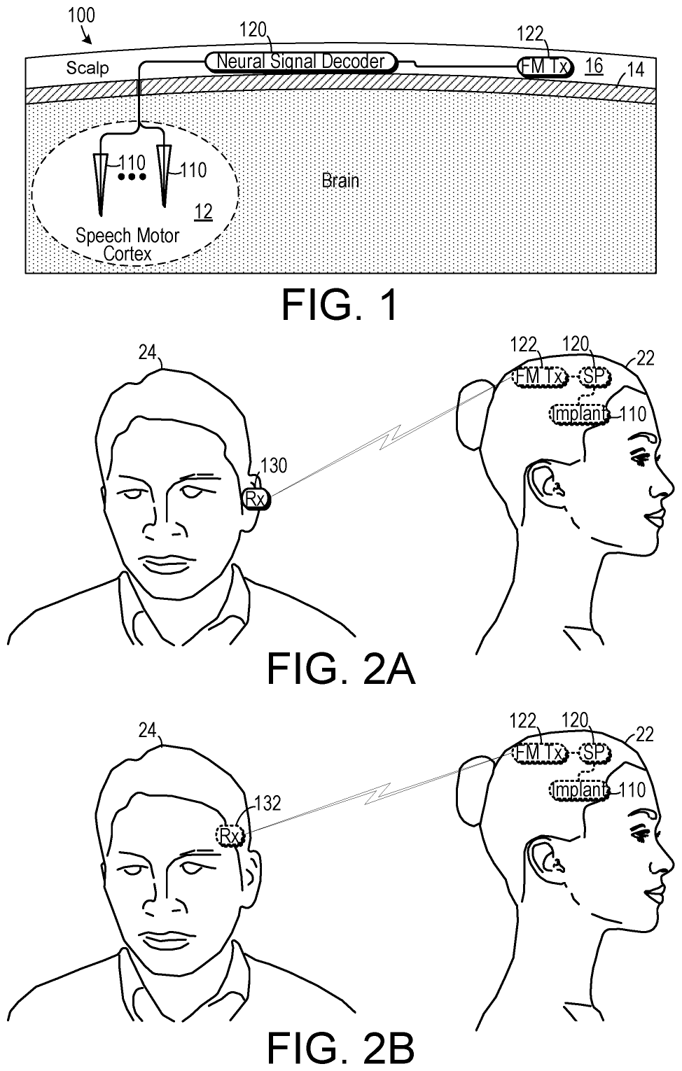

As shown in FIG. 1, one embodiment of a silent speech generation system 100 includes at least one neural implant 110 implanted into the speech motor cortex 12 of a first person. Speech neural impulses are generated by the first person while silently generating neural motor potentials corresponding to speech. (Speaking silently means attempting to move the muscles in the mouth and the muscles controlling the vocal cords without generating sound. Such silent speaking requires the speech motor cortex to generate corresponding neural motor potentials, which are activating neural signals to the muscles.) The neural motor potentials are sensed by the implants 110, which are sent to a neural signal decoder 120 that generates an electrical signal that corresponds to phones, phonemes, words or phrases that correlate to the neural motor potentials. The electrical signal is modulated onto a radio-frequency signal that is transmitted by a transmission circuit 122. The neural signal decoder 120 and the transmission circuit 122 can be embedded into the first person's scalp. Communication can be effected, for example, in one embodiment by using existing personal area network systems (e.g., BlueTooth, Zigbee, etc.)

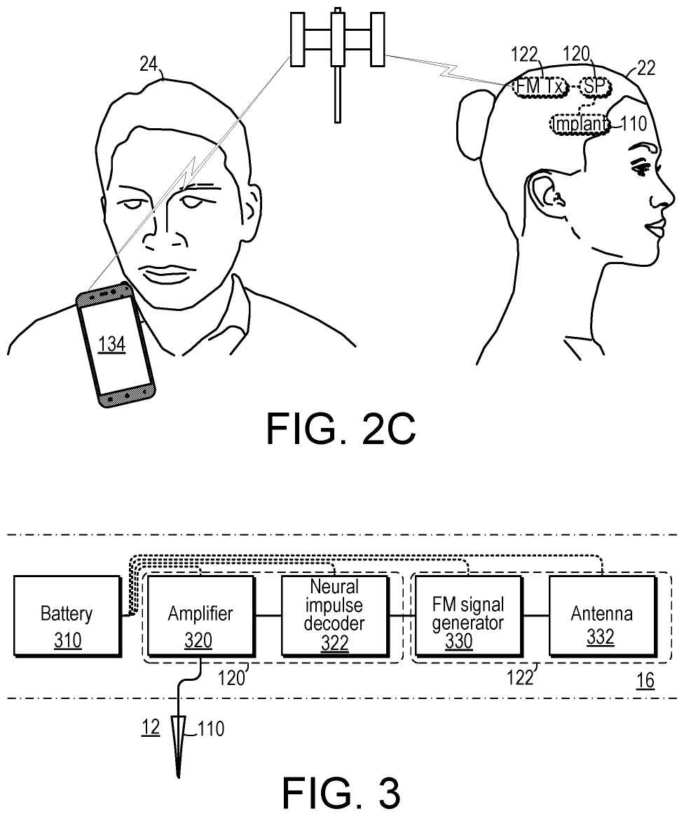

As shown in FIG. 2A, when the first person 22 speaks silently, the silent speech generation system 100 wirelessly transmits the signal to the second person 24, who receives a wireless signal (such as an FM signal) from the first person 22 with a receiver. In the embodiment shown, the wireless signal can be received with a hearing aid 130 that includes a wireless receiver that converts the wireless signal into an audible signal. As shown in FIG. 2B, the receiver can include an implant 132, such as a sound generating device (using hearing aid electronics in one embodiment) implanted into the second person's mastoid bone. Alternately, as shown in FIG. 2C, the transmission circuit 122 can include a cellular chipset and the receiver can include a cellular telephone 134. In this embodiment, the first person can talk audibly or silently to use the system essentially as an implanted cellular telephone.

As shown in FIG. 3, the speech generation system 100 can include a battery 310 that powers the system. An amplifier 320 (such as an op-amp) amplifies the potentials sensed by the neural implant 110 and a neural impulse decoder 322 (which can include, for example, a neural network, such as a convolutional neural network trained to correlate neural potentials to elements of speech) transforms the amplified potentials into an electronic signal corresponding to phones, phonemes, words or phrases. The electronic signal is transformed into a radio-frequency signal (e.g., an FM signal) by a radio signal generator 330, which is then transmitted by an antenna 332.

Experimentally, it has been determined that a human brain to a speech prosthetic interface can provide at least 100 useful words or phrases at a near conversational rate. The implantable neurotrophic electrode, employed in one embodiment, is based on the ingrowth of neuropil into its 1.5 mm glass tip thereby securing the neural signals for long-term recording. Recent data show that the neurotrophic electrode used for the interface provides long lasting recordings that were functional years after implantation. Alternate embodiments can employ external electrode placement, such as: electroencephalographic (EEG) sensors, brain surface sensors (Electrocorticography, ECOG) and subsurface recordings such as with Tine type electrodes.

Invasive type electrodes can record from single units whose firing patterns are thought to closely reflect the underlying cortical function. The pattern of firings during overt speech map fairly closely to those used during covert speech.

The neural electrodes are constructed as follows: 2 mil teflon insulated gold wires are coiled around a pipette and glued with methyl-methacrylate inside a glass cone. The cone is made by pulling a heated pipette and obtaining the tip to the dimensions required which are 1.5 mm in length, 25 microns at the deep end and a few hundred microns at the upper end to allow space for the inserted wires. The other end of each coiled gold wire is soldered into a connector that will plug into the implanted electronic component.

In one experimental embodiment three single channel amplifiers were implanted. The amplifiers were bipolar amplifiers in record pairs of wires via the low impedance (50 to 500 kOhms) gold wires that were cut across the tip to provide low impedance recordings. These were connected to an FM transmitter operating in the carrier 35 to 55 MHz range. The amplifier had a gain of 100× and is filtered between 5 and 5,000 Hz. During experimental recording sessions, a power induction coil powered the device with the induced current passing through a regulator to provide +/−3 volts. The electronics were insulated with a polymer (Elvax: Ethylene Vinyl Acetate Copolymer Resin, from DuPont, Wilmington, Del. 19898) and further insulated (and protected against trauma) with Silastic (Med-6607, Nusil, Silicone Technology, Carpentaria, Calif.). The gold pin connection to the electrodes was protected with acrylic cement (Medtronic Inc., St. Paul, Minn.). The whole implant was covered with scalp skin. Three of the eight pairs of electrodes wires were attached to three sets of connecting pins that were, in turn, attached to three electronic amplifiers and FM transmitters.

In the experimental embodiment, two cones with a total of four wires were inserted in the subject. It was determined that neurons outside of the tip extended as neurites into the implanted glass cone. The neurites become myelinated within three weeks. Four electrode tips were implanted 6 mm apart. Each electrode tip contained four wires. Three pairs of wires were attached to three sets of connector pins. These pins were attached to three devices that were attached to three power induction coils.

Prior to the implantation procedures, functional MRI was employed to localize areas of articulation in the subject. Articulatory movements consisted of protrusion and retraction of the tongue, jaw closing and opening, and cheek grinning and pouting. The speech motor area is localized 3 cm medial to the Sylvian fissure in primary motor cortex. Operation of this device is a motor task since it is the neural signals associated with movement of the articulators that are recorded.

Single channel FM transmitters were implanted. The implanted recording amplifiers had gains of 100× with a bandpass filter of 5 to 5,000 Hz. The FM receivers (WinRadio Inc, Oakleigh, Australia) were tuned to the FM frequencies in the range of 35 to 55 MHz. An external amplifier for each channel (BMA-200, CWE Inc. Ardmore, Pa.) had a gain of 100× , with a bandpass filter of 1 to 10,000 Hz.

Using fast Fourier transforms of the continuous signals also contributed to the results. Beta peaks (defined as 12 to 20 Hz increases above baseline in a ratio of 100:1) and the event markers are used to determine the onset of speech.

Initial experimental analysis focused on five phonemes that had the most single unit activity associated with them (Bead (‘eeh’), Chin (‘ch’), Go (‘guh’), Judge (‘juh’), Fine (‘feh’)). Single unit bursts (and consequent quiet periods) were assessed over the time in which the phoneme was being spoken overtly or covertly. The bursts were scored by visual examination according to their height and trough as plus or minus, independent of their duration or the amplitude of their height or trough. Scoring consisted of 1=an increase or decrease in firing modulation; 2=an increase followed by a decrease; 3=a decrease followed by an increase; 4=increase followed by a decrease followed by an increase; 5=a decrease followed by an increase followed by a decrease. This analysis was completed for each of the 23 single units in electrode 3, for all 10 trials within each session and for each of the five phonemes (Bead, Chin, Go, Judge, Fine). The data were tabulated, and the commonest scores were noted for each single unit. These data formed a pattern of unique activity across all single units. Using these patterns of single unit firings, a proprietary software program was used to detect these patterns. The paradigm involved searching for average activity prior to and following the burst of activity. The amount of time allotted for this prior and post burst calculation was a time bin of 50, 20, 10, 5 or 1 ms.

A simple Fitting architecture was used to map from a set of inputs to a corresponding set of outputs (Fitting function in Matlab, (Natwick, Mass.)). The inputs were patterns of neural bursts and the outputs are known or estimated patterns of neural bursts. The standard neural network architecture for fitting problems is a multilayer perceptron. The Tansig function is used in the middle layers that outputs to the next layer. The advantage of the Tansig function is that it is centered around zero and not always positive since its values vary between −1 and 1. The alternatively used Logsig transfer function is always positive and this would lose half the data because its values vary between 0 and 1. A hidden layer of 1 or 2 and sometimes 10 is usually all that is required. The number of layers is chosen by trial and error. Thus, the initial step is to determine the likely single unit firing pattern by examining the burst patterns of the single units as described above. This typical pattern is designated as the target output. Standard patterns of firing for each word or phrase are then catalogued (which can be viewed as a ‘look up table’) and used for speech production as new neural firing inputs are transferred through the system. The algorithms used in the fitting app were the standard Levenberg-Marquardt, Bayesian Regularization and Scaled Conjugate Gradient.

The data from the speech area of subject of the experimental embodiment indicate that single units can be recorded and incorporated into analytic programs that detect components of speech including phonemes, words and phrases. Individual single unit bursts demonstrate that phones, phonemes, words and phrases can be recovered from the patterns of firings using the proprietary software program as well as from artificial neural net paradigms, such as the Fitting program.

One training method employed the following steps:
1. Obtain recordings from the subject while ‘speaking’ covertly. The subject will repeat the same phrase at least 10 times. The subject will then repeat different phrases such as ‘Hello’, ‘How are you?’ and so on at least 10 times each.
2. Separate the multi-units into single units using, e.g., Neuralynx's Cheetah program.
3. Detect the Beta peaks using a software program called Beta Peak Detection to determine covert speech onset.
4. Classify the burst firing pattern of the single units.
5. Average the burst patterns to produce the Target for the Neural Net
6. Fitting program.
7. Enter the averaged results into the Fitting program to form the Targets for the Fitting program.
8. As the subject covertly speaks online, the Beta Peak Detection program will recognize the onset of the covert speech.
9. The single unit bursts are detected and classified using the Classify program. This will tag the bursts as 0, 1, 2, 3, 4 or 5 and form the Input data.
10. During covert speech the Input data bursts (0, 1, 2, 3, 4 or 5) interact with the already established Target of the Fitting program allowing it to recognize the words or phrases from the patterns 0, 1, 2, 3, 4 or 5.
11. With word or phrase recognition, the Fitting program output selects the appropriate wave file containing that word or phrase and emits it from the computer speaker. The subject will hear the word or phrase and repeat if necessary to improve recognition.

In one representative embodiment, the first person speaks silently via FM to the second person. Hearing aids can receive the resulting FM transmission. The hearing aid can be embedded in the mastoid bone behind the ear. It can be wirelessly powered using WattUp from Energous Inc. The WattUp chip inside is 2 mm×2 mm and charges a battery. The battery charges the hearing aid and the electronics with a sub-scalp lead from the mastoid bone to the electronics under the scalp. The second person hears and understands and then speaks silently in reply (in a situation in which both the first person and the second person are both implanted with silent speech generating units). The electrodes are implanted in the motor speech area that is in primary motor cortex just inches above the left ear in left dominant hemispheres.

Once decoded, the signals are FM transmitted back to the first person. The first person can have a similar implanted system for bidirectional silent communication.

The initial implant will involve only the electrodes. The brain's neuropil takes three months to grow into the hollow tip of the electrode. Once that is achieved, the electrode leads will be externalized for a week or two to enable decoding. The first step in decoding is to separate the single units from the continuous stream of data. The second step is to have the subject repeat useful words or short phrases 10 or more times so that the system can build a library of 100 or more words and phrases based on the pattern of firing of 100s of single units. Streaming of single units past the library will match a word or phrase and that word or phrase will be made audible by triggering a wave file containing that word. Once that is accomplished, the single unit parameters and the subsequent decoding will be placed on an application-specific integrated circuit (ASIC) chip. The output of the chip will be attached to an FM transmitter. In this embodiment, the amplifiers, single unit streaming, decoding and building of library, wave-file and FM transmitter will be implanted back under the scalp of the individual.

This system can be powered by a hearing aid/power system which is implanted at the same time with the power lead traveling under the scalp to the electronic system. The hearing aid that receives the FM signal is modified with the WattUp receiving chip attached to the battery. A power wire is inserted to carry power from the hearing aid to the electronics.

The neurotrophic electrode, that provides growth of brain into the tip, has provided stable and functional signals for more than a decade in human subjects. This is because the brain neuropil grows through both ends of the tip, becomes myelinated and anchors the tip within the cortex. The Teflon insulated 99.999% gold wires are coiled to provide strain relief. Histological analysis of the tissue within tip shows no scarring, i.e., no gliosis, and myelinated neurafilaments confirming that recordings were from neural tissue.

Off-line analysis indicates that silent speech (as defined above) can be detected with no significant difference between audible speech and silent speech.

While the embodiments above discuss use of the system for silent communication between individuals, the invention can also be used to allow communication by locked in patients.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A silent communication system for communication between a first person having a speech motor cortex and a second person, comprising:
(a) a speech motor cortex neural sensing device configured to sense speech neural impulses generated by the first person when the first person is generating motor neural potentials corresponding to speech;
(b) a wireless transmission device configured to be disposed on the first person that is in communication with the speech motor cortex neural sensing device and that generates a radio frequency signal corresponding to the speech neural impulses; and
(c) a speech generating device disposed on the second person that is responsive to the radio frequency signal, the speech generating device configured to generate a reconstruction of the speech of the first person that is audibly perceptible by the second person.

2. The silent communication system of claim 1, wherein the speech motor cortex neural sensing device comprises:
(a) at least one neural implant configured to be implanted into the speech motor cortex of the first individual;
(b) a signal processor that is responsive to the neural implant that decodes the neural impulses and that generates an electrical signal that synthesizes the speech of the first person,
wherein the wireless transmission device is responsive to the electrical signal.

3. The silent communication system of claim 1, wherein the speech generating device comprises a signal processor that that decodes the neural impulses and that generates an electrical signal that synthesizes the speech of the first person.

4. The silent communication system of claim 1, wherein the wireless transmission device comprises a FM transmitter.

5. The silent communication system of claim 1, wherein the speech generating device comprises:
(a) a radio frequency receiver that is responsive to the radio frequency signal; and
(b) an electronic hearing aid, configured to be worn by the second person, that is in communication with the radio frequency receiver and that generates an audible signal corresponding to the radio frequency signal.

6. The silent communication system of claim 1, wherein the speech generating device comprises:
(a) a radio frequency receiver that is responsive to the radio frequency signal; and
(b) a sound generating device, configured to be implanted into the mastoid bone of the second person, that is in communication with the radio frequency receiver and that generates an audible signal corresponding to the radio frequency signal.

7. The silent communication system of claim 1, wherein the wireless transmission device comprises a cellular telephone chipset and wherein speech generating device comprises a cellular telephone.

8. A method of communicating silently between a first person having a speech motor cortex and a second person, comprising the steps of:
(a) sensing speech neural impulses from the speech motor cortex of the first person when the person generates motor neural potentials corresponding to speech;
(b) generating an electrical signal corresponding to synthesized speech using the speech neural impulses as input;
(c) modulating the electrical signal onto a radio frequency signal;
(d) transmitting the radio frequency signal;
(e) receiving the radio frequency signal; and
(f) generating reconstructed speech that is audibly perceptible by the second person from the radio frequency signal.

9. The method of claim 8, further comprising the step of implanting at least one neural implant into the speech motor cortex of the first person and wherein the step of sensing speech neural impulses comprises receiving the neural impulses from the implant.

10. The method of claim 8, wherein the step of generating an electrical signal corresponding to synthesized speech comprises the steps of:
(a) correlating the neural impulses to phones, phonemes, words or phrases;
(b) synthesizing the phones, phonemes, words or phrases into the electrical signal.

11. The method of claim 8, wherein the radio frequency signal comprises a frequency modulated signal.

12. The method of claim 8, wherein the reconstructed speech is heard by the second person through a hearing aid.

13. The method of claim 8, further comprising the step of implanting a cochlear implant into the second person and wherein the reconstructed speech is heard by the second person through the cochlear implant.

14. The method of claim 8, wherein the step of transmitting the radio frequency signal comprises transmitting a cellular telephone signal and wherein the reconstructed speech is heard by the second person through a cellular telephone.

15. A method of communicating silently between a first person having a speech motor cortex and a second person, comprising the steps of:
(a) sensing speech neural impulses from the speech motor cortex of the first person when the person generates motor neural potentials corresponding to speech;
(b) generating an electrical signal corresponding to the speech neural impulses;
(c) modulating the electrical signal onto a radio frequency signal;
(d) transmitting the radio frequency signal;
(e) receiving the radio frequency signal; and
(f) demodulating the radio frequency signal;
(g) decoding the speech neural impulses into phones, phonemes, words or phrases; and
(h) generating from the phones, phonemes, words or phrases reconstructed speech that is audibly perceptible by the second person from the radio frequency signal.

16. The method of claim 15, further comprising the step of implanting at least one neural implant into the speech motor cortex of the first person and wherein the step of sensing speech neural impulses comprises receiving the neural impulses from the implant.

17. The method of claim 15, wherein the radio frequency signal comprises a frequency modulated signal.

18. The method of claim 15, wherein the reconstructed speech is heard by the second person through a hearing aid.

19. The method of claim 15, further comprising the step of implanting a cochlear implant into the second person and wherein the reconstructed speech is heard by the second person through the cochlear implant.

20. The method of claim 15, wherein the step of transmitting the radio frequency signal comprises transmitting a cellular telephone signal and wherein the reconstructed speech is heard by the second person through a cellular telephone.
